(19) United States
(12) Patent Application Publication
(10) Pub. No.: US 2016/0089441 A1
(43) Pub. Date: Mar. 31, 2016

(54) COMPOSITION FOR THE TREATMENT OF COLD SORES

(71) Applicant: ORALABS, INC., Parker, CO (US)

(72) Inventors: Christopher P. Turner, Parker, CO (US); Gary H. Schlatter, Englewood, CO (US)

(21) Appl. No.: 14/865,834

(22) Filed: Sep. 25, 2015

Related U.S. Application Data
(60) Provisional application No. 62/055,254, filed on Sep. 25, 2014.

(51) Int. Cl. A61K 47/26; A61K 47/10; A61K 31/245; A61K 47/22; A61K 31/045; A61K 9/06; A61K 47/06

(52) U.S. Cl. CPC A61K 47/26; A61K 9/06; A61K 47/10; A61K 47/06; A61K 47/22; A61K 31/045; A61K 31/245

(57) ABSTRACT

A composition used in the treatment of the cold sores is composed of Water, Propylene Glycol, Mineral Oil, Behenyl Alcohol or docosanol, Benzocaine, Vitamin E tocopheryl, Sucrose Stearate, and Benzyl Alcohol.

### CROSS REFERENCE TO RELATED APPLICATION

This application claims the benefit of U.S. Provisional Patent Application Ser. No. 62/055,254, entitled “COMPOSITION FOR THE TREATMENT OF COLD SORES,” filed Sep. 25, 2014.

### BACKGROUND OF THE INVENTION

1. Field of the Invention

The present invention relates to a composition for the treatment of cold sores.

2. Description of the Related Art

Cold sores (herpes labialis) commonly result in small sores and/or blisters forming about and within a victims mouth. Cold sores are commonly believed to be caused by a certain type of the herpes simplex virus. This type of virus usually affects the mouth, causing recurrent sores on the lips and areas around the mouth. On occasion, this virus gets into the blood stream and causes a serious infection in babies.

There are currently there are a number of medications commercially available that attempt to treat the herpes virus and its symptomatic sores or blisters. Some are designed to quickly kill the virus, and are therefore somewhat hard on the skin and can cause irritation to a treated lesion and surrounding tissue. Others medications are mostly topical, and fail to provide a mechanism to assist active agents to penetrate the skin and provide deep healing effects. Still others are designed to provide a soothing effect, but fail to effectively combat and kill the virus, which leads to reduced symptoms, but does not make the infection clear up quickly.

Given the discomfort, pain and unpleasant appearance of cold sores there exists a need for effective methods for the treatment of the cold sores.

### SUMMARY OF THE INVENTION

It is, therefore, an object of the present invention to provide a composition used in the treatment of the cold sores. The composition is composed of Water, Propylene Glycol, Mineral Oil, Behenyl Alcohol or docosanol, Benzocaine, Vitamin E tocopheryl, Sucrose Stearate, and Benzyl Alcohol.

It is also an object of the present invention to provide a composition wherein the composition is a cream.

It is another object of the present invention to provide a composition wherein the composition is composed of 50%-90% by weight Water, 2%-10% by weight Propylene Glycol, 1%-10% by weight Mineral Oil, 1%-50% by weight Behenyl Alcohol, 5%-20% by weight Benzocaine, 0%-1% by weight Vitamin E, 1%-10% by weight Sucrose Stearate and 1%-10% by weight Benzyl Alcohol.

It is a further object of the present invention to provide a composition wherein the composition is composed of 64% by weight Water, 4.99% by weight Propylene Glycol, 2% by weight Mineral Oil, 10% by weight Behenyl Alcohol, 5% by weight Benzocaine, 0.01% by weight Vitamin E, 5% by weight Sucrose Stearate and 9% by weight Benzyl Alcohol.

It is also an object of the present invention to provide a composition used in the treatment of the cold sores and made in accordance with a method comprising mixing Water and Propylene Glycol and mixing Mineral Oil, Behenyl Alcohol, Benzocaine, Sucrose Stearate and Vitamin E. The mixture of Water and Propylene Glycol and the mixture of Mineral Oil, Behenyl Alcohol, Benzocaine, Sucrose Stearate and Vitamin E are then combined. Benzyl alcohol is added to the mixture of Water and Propylene Glycol and the mixture of Mineral Oil, Behenyl Alcohol, Benzocaine, Sucrose Stearate and Vitamin E.

It is another object of the present invention to provide a composition wherein Mineral Oil, Behenyl Alcohol, Benzocaine, Sucrose Stearate and Vitamin E are mixed at a temperature of 150° F.-300° F. for no less than 25 minutes under agitation.

It is a further object of the present invention to provide a composition wherein the mixture of Water and Propylene Glycol and the mixture of Mineral Oil, Behenyl Alcohol, Benzocaine, Sucrose Stearate and Vitamin E are respectively set to a temperature of 140° F.-180° F. prior to mixing.

It is also an object of the present invention to provide a composition wherein the step of adding Benzyl alcohol to the mixture of Water and Propylene Glycol and the mixture of Mineral Oil, Behenyl Alcohol, Benzocaine, Sucrose Stearate and Vitamin E is followed by homogenizing for 10-20 minutes while maintaining a temperature of 140° F.-180° F.

Other objects and advantages of the present invention will become apparent from the following detailed description when viewed in conjunction with the accompanying drawings, which set forth certain embodiments of the invention.

### DESCRIPTION OF THE PREFERRED EMBODIMENT

The detailed embodiment of the present invention is disclosed herein. It should be understood, however, that the disclosed embodiment is merely exemplary of the invention, which may be embodied in various forms. Therefore, the details disclosed herein are not to be interpreted as limiting, but merely as a basis for teaching one skilled in the art how to make and/or use the invention.

As discussed above, the present invention relates to a composition used in the treatment of the cold sores. Briefly, the cold sore treatment composition is prepared in the form of a cream. Although a cream is disclosed and described herein, it is appreciated the character of the delivery composition may be varied to suite specific applications. This combination results in a cold sore treatment composition for topical application so as to effective treat cold sores once they appear.

Briefly, and in accordance with a preferred embodiment of the present invention, the cold sore treatment composition includes Water, Propylene Glycol, Mineral Oil, Behenyl Alcohol or docosanol, Benzocaine, Vitamin E tocopheryl, Sucrose Stearate and Benzyl Alcohol.

Considering the functional aspects of the present invention, the composition includes a physiologically active amount of Benzocaine and Behenyl Alcohol working with the remaining various inactive delivery components to achieved a synergistic effect in the treatment of cold sores. Physiologically active ingredients and the delivery components are therefore composed of Water, Propylene Glycol, Mineral Oil, Behenyl Alcohol, Benzocaine, Vitamin E or tocopherol, Sucrose Stearate and Benzyl Alcohol. These components are mixed in the following manner to achieve a composition in accordance with the present invention. First, Water and Propylene Glycol are mixed to produce Phase 1. Then Mineral Oil, Behenyl Alcohol, Benzocaine, Sucrose Stearate and Vitamin E are mixed at a temperature of 150° F.-300° F. for no less than 25 minutes under agitation to optimize physiological delivery of the drug and produce Phase 2. Phase 1 and Phase 2 are set to a temperature of 140° F.-180° F. and then combined. Benzyl alcohol is then added and the formula is homogenized for 10-20 minutes while maintaining a temperature of 140° F.-180° F. It is appreciated the specific order of mixing is important in that some components are more stable than others and mixing in this manner allows for the components to maintain their effectiveness in accordance with the purposes of the present invention.

In accordance with a preferred embodiment, the composition is composed of 50%-90% by weight Water, 2%-10% by weight Propylene Glycol, 1%-10% by weight Mineral Oil, 1%-50% by weight Behenyl Alcohol, 5%-20% by weight Benzocaine, 0%-1% by weight Vitamin E, 1%-10% by weight Sucrose Stearate and 1%-10% by weight Benzyl Alcohol. More preferably, the composition is composed of 64% by weight Water, 4.99% by weight Propylene Glycol, 2% by weight Mineral Oil, 10% by weight Behenyl Alcohol, 5% by weight Benzocaine, 0.01% by weight Vitamin E, 5% by weight Sucrose Stearate and 9% by weight Benzyl Alcohol.

The present composition when applied to an infected area exerts antiviral activities killing the virus responsible for the cold sores and resulting in a more rapid clearing of the sores from the skin of the individual affected thereby. The composition is repeatedly applied to the cold sore daily. The duration of these daily treatments can last for anywhere from 3 to 7 days, depending on the severity of the cold sore and related symptoms. Once the cold sore is in remission and no longer visible, administration of the composition can be terminated. If left alone, most cold sores heal on their own in 7 to 10 days, says the Mayo Clinic. One to two days before watery blisters erupt on or near the lips or mouth, you may experience a painful or tingling sensation on the skin. This is the best time to apply the composition of the present invention. It is also best to apply the composition before blisters rupture. Once blisters rupture the composition is still applied to aiding healing without scarring. The composition of the present invention has been found to reduce the 7 to 10 day period in most of the population.

While the preferred embodiments have been shown and described, it will be understood that there is no intent to limit the invention by such disclosure, but rather, is intended to cover all modifications and alternate constructions falling within the spirit and scope of the invention.

1. A composition used in the treatment of the cold sores, comprising:
Water;
Propylene Glycol;
Mineral Oil;
Behenyl Alcohol or docosanol;
Benzocaine;
Vitamin E tocopheryl;
Sucrose Stearate; and
Benzyl Alcohol.

2. The composition according to claim 1, wherein the composition is a cream.

3. The composition according to claim 2, wherein the composition is composed of 50%-90% by weight Water, 2%-10% by weight Propylene Glycol, 1%-10% by weight Mineral Oil, 1%-50% by weight Behenyl Alcohol, 5%-20% by weight Benzocaine, 0%-1% by weight Vitamin E, 1%-10% by weight Sucrose Stearate and 1%-10% by weight Benzyl Alcohol.

4. The composition according to claim 2, wherein the composition is composed of 64% by weight Water, 4.99% by weight Propylene Glycol, 2% by weight Mineral Oil, 10% by weight Behenyl Alcohol, 5% by weight Benzocaine, 0.01% by weight Vitamin E, 5% by weight Sucrose Stearate and 9% by weight Benzyl Alcohol.

5. The composition according to claim 1, wherein the composition is composed of 50%-90% by weight Water, 2%-10% by weight Propylene Glycol, 1%-10% by weight Mineral Oil, 1%-50% by weight Behenyl Alcohol, 5%-20% by weight Benzocaine, 0%-1% by weight Vitamin E, 1%-10% by weight Sucrose Stearate and 1%-10% by weight Benzyl Alcohol.

6. The composition according to claim 1, wherein the composition is composed of 64% by weight Water, 4.99% by weight Propylene Glycol, 2% by weight Mineral Oil, 10% by weight Behenyl Alcohol, 5% by weight Benzocaine, 0.01% by weight Vitamin E, 5% by weight Sucrose Stearate and 9% by weight Benzyl Alcohol.

7. A composition used in the treatment of the cold sores, made in accordance with a method comprising:
mixing Water and Propylene Glycol;
mixing Mineral Oil, Behenyl Alcohol, Benzocaine, Sucrose Stearate and Vitamin E;
combining the mixture of Water and Propylene Glycol and the mixture of Mineral Oil, Behenyl Alcohol, Benzocaine, Sucrose Stearate and Vitamin E;
adding Benzyl alcohol to the mixture of Water and Propylene Glycol and the mixture of Mineral Oil, Behenyl Alcohol, Benzocaine, Sucrose Stearate and Vitamin E.

8. The composition according to claim 7, wherein Mineral Oil, Behenyl Alcohol, Benzocaine, Sucrose Stearate and Vitamin E are mixed at a temperature of 150° F.-300° F. for no less than 25 minutes under agitation.

9. The composition according to claim 8, wherein the mixture of Water and Propylene Glycol and the mixture of Mineral Oil, Behenyl Alcohol, Benzocaine, Sucrose Stearate and Vitamin E are respectively set to a temperature of 140° F.-180° F. prior to mixing.

10. The composition according to claim 7, wherein the step of adding Benzyl alcohol to the mixture of Water and Propylene Glycol and the mixture of Mineral Oil, Behenyl Alcohol, Benzocaine, Sucrose Stearate and Vitamin E is followed by homogenizing for 10-20 minutes while maintaining a temperature of 140° F.-180° F.

11. The composition according to claim 10, wherein the composition is composed of 50%-90% by weight Water, 2%-10% by weight Propylene Glycol, 1%-10% by weight Mineral Oil, 1%-50% by weight Behenyl Alcohol, 5%-20% by weight Benzocaine, 0%-1% by weight Vitamin E, 1%-10% by weight Sucrose Stearate and 1%-10% by weight Benzyl Alcohol.

12. The composition according to claim 10, wherein the composition is composed of 64% by weight Water, 4.99% by weight Propylene Glycol, 2% by weight Mineral Oil, 10% by weight Behenyl Alcohol, 5% by weight Benzocaine, 0.01% by weight Vitamin E, 5% by weight Sucrose Stearate and 9% by weight Benzyl Alcohol.

13. The composition according to claim 7, wherein the mixture of Water and Propylene Glycol and the mixture of Mineral Oil, Behenyl Alcohol, Benzocaine, Sucrose Stearate and Vitamin E are respectively set to a temperature of 140° F.-180° F. prior to mixing.

14. The composition according to claim 7, wherein the step of adding Benzyl alcohol to the mixture of Water and Propylene Glycol and the mixture of Mineral Oil, Behenyl Alcohol, Benzocaine, Sucrose Stearate and Vitamin E is followed by homogenizing for 10-20 minutes while maintaining a temperature of 140° F.-180° F.

15. The composition according to claim 7, wherein the composition is a cream.

16. The composition according to claim 7, wherein the composition is composed of 50%-90% by weight Water, 2%-10% by weight Propylene Glycol, 1%-10% by weight Mineral Oil, 1%-50% by weight Behenyl Alcohol, 5%-20% by weight Benzocaine, 0%-1% by weight Vitamin E, 1%-10% by weight Sucrose Stearate and 1%-10% by weight Benzyl Alcohol.

17. The composition according to claim 7, wherein the composition is composed of 64% by weight Water, 4.99% by weight Propylene Glycol, 2% by weight Mineral Oil, 10% by weight Behenyl Alcohol, 5% by weight Benzocaine, 0.01% by weight Vitamin E, 5% by weight Sucrose Stearate and 9% by weight Benzyl Alcohol.
